(19) United States
(12) Patent Application Publication
(10) Pub. No.: US 2003/0223950 A1
(43) Pub. Date: Dec. 4, 2003

(54) Water-free hairstyling gels

(76) Inventors: Michael Lede, Langen (DE); Susanne Birkel, Darmstadt (DE); Beate Grasser, Hattersheim (DE); Walter Keller, Ober-Ramstadt (DE)

Correspondence Address: STRIKER, STRIKER & STENBY, 103 East Neck Road, Huntington, NY, 11743, US

(21) Appl. No.: 10/423,405

(22) Filed: Apr. 25, 2003

(30) Foreign Application Priority Data
May 2, 2002 (DE) 102 19 597.8

(51) Int. Cl. A61K 7/06; A61K 7/11

(52) U.S. Cl. 424/70.15; 424/70.16; 424/70.17

(57) ABSTRACT

The hairstyling gel is formulated on an alcoholic, substantially water-free basis and contains a combination of an alcohol-soluble, film-forming and hair-fixing polymer, a gel-former, which is in a position to thicken a liquid alcohol, and more than 30 percent by weight of a C₁- to C₄-alcohol.

### BACKGROUND OF THE INVENTION

1. Field of the Invention

The subject matter of the present invention are hairstyling gels on an alcoholic, substantially water-free basis, which contain a combination of an alcohol-soluble, film-forming and hair-fixing polymer, a gel former which is in a position to thicken liquid alcohols and more than 30 percent by weight of a C₁- to C₄-alcohol.

2. Description of the Related Art

Hair treatment compositions, among others, in the form of hair gels, are used in order to fix and hold human hair. Hair gels are usually characterized by good distributability and workability in the hair. The hair may be in the form of a hairstyle, which is fixed after drying the gel. Conventional hair gels principally comprise water, a water-soluble thickening system and water-soluble styling polymer (Harry's Cosmetology, Chap. 8, pp. 647-650). In exceptional cases a small amount of alcohol can be added, which however does not change the basic microscopic and macroscopic structure of the gel, since the much greater amount of water in the gel outweighs the small amount of alcohol. The conventional gels based on water are not always completely satisfactory. They have the disadvantage that they require a very long drying time after application. Moreover the water-soluble styling polymers that can be included in the hairstyling gels on a water basis and their inherent properties are limited. All desirable application properties, for example regarding fixing degree, moisture resistance, shaping action, feel, elasticity, protection or luster, of the treated hair, could not be obtained. Limits are put on the optimization of the action profile.

### SUMMARY OF THE INVENTION

Thus it is an object of the present invention to provide a hair treatment composition, which has the application advantages of a conventional hair gel, but at the same time has an expanded or improved action profile.

According to the invention it has now been found that this object is attained by a hair gel containing a combination of

(A) at least one alcohol-soluble, film-forming and hair-fixing polymer;

(B) at least one gel-former capable of thickening, or in a position to thicken, liquid alcohols; and

(C) more than 30 percent by weight of a C₁- to C₄-alcohol.

The polymer (A) is contained in the hair gel preferably in an amount of from 0.1 to 20, especially preferably from 0.5 to 15, and most preferably from 1 to 10, percent by weight. The gel former (B) is contained preferably in an amount of 0.01 to 20, especially preferably from 0.05 to 10 and most preferably from 0.1 to 7, percent by weight. The alcohol (C) is preferably present in the hair gel in an amount of from 50 to 99, especially preferably from 70 to 98, percent by weight.

The term “alcohol-soluble polymer” means a polymer, which is soluble to at least five percent by weight in at least one alcohol with 1 to 4 carbon atoms at 25° C. The term “film-forming and hair-fixing polymer” means a polymer, which exerts a hair-fixing action, especially increases curl retention in relation to untreated hair, when used in a 0.01 to 5% solution.

Suitable hair-fixing polymers according to the invention can be nonionic, anionic, cationic, zwitterionic or amphoteric. They can include synthetic or natural polymers. The term “natural polymer” also includes chemically modified polymers of natural origin. Copolymers made from at least one first monomer, which is acrylic acid and/or methacrylic acid, and at least one second monomer selected from the group consisting of acrylic acid esters, methacrylic acid esters, acrylamides and methacrylamide. The esters also can be alkyl-, aminoalkyl-, monoalkylaminoalkyl or dialkylaminoalkyl esters. The amides can be unsubstituted amides, monoalkylamides, dialkylamides, alkylaminoalkylamides or (dialkylamino)alkylamides. The alkyl groups mentioned above preferably have 1 to 30, especially preferably 1 to 5, carbon atoms. Additional suitable hair-fixing polymers include copolymers of crotonic acid and at least one nonionic comonomer. The nonionic comonomers can be the above-mentioned (methyl)acryl ester, (meth)acryl amide or vinyl ester. Suitable vinyl esters include the esters of vinyl alcohol and C₁- to C₁₂-carboxylic acid. Additional suitable hair-fixing polymers include copolymers of at least one acrylic or methacrylic acid ester and at least one nonionic comonomer. The nonionic comonomer can be the above-described (meth)acrylic ester or (meth)acrylic amide, the above-mentioned vinyl ester or vinyl lactam, e.g. vinyl pyrrolidone or vinyl caprolactam. Additional suitable hair-fixing polymers are copolymers of at least one maleic acid monoester and at least one nonionic comonomer. A C₁- to C₇ alkyl ester of maleic acid is suitable as the maleic acid monoester. Suitable nonionic comonomers are those mentioned above or alkylvinyl ether, especially methyl vinyl ether. Polyvinyl caprolactams are additional suitable hair-fixing polymers. Additional suitable hair-fixing polymers are copolymers of at least one first monomer, which is a vinyl lactam, such as vinyl caprolactam, or vinyl pyrrolidone, and at least one second monomer, which is a suitable vinyl ester, especially vinyl acetate.

Preferred hair-fixing polymers include polyvinyl caprolactam (e.g. LUVISKOL® PLUS), vinylpyrrolidone/vinyl acetate copolymer (e.g. LUVISKOL®VA 37), terpolymers of vinylpyrrolidone, vinyl acetate and vinyl propionate (e.g. LUVISKOL®VAP 343), copolymers of acrylic acid or methacrylic acid with monomers selected from the group consisting of acrylic acid esters, methacrylic acid esters, acrylic acid amides and methacrylic acid amides, especially acrylic acid/ethylacrylate/N-tert-butylacrylate copolymer (e.g. ULTRAHOLD® 8), octylacrylamide/acrylic acid/t-butylaminoethylmethacrylate/hydroxypropylmethacrylate copolymer (e.g. AMPHOMER®), t-butyl-acrylate/ethylacrylate/methacrylic acid copolymer (e.g. LUVIMER® 100P), vinylacetate/crotonic acid copolymer (e.g. LUVISET® CA66 or RESYN 28 1310), vinyl acetate/crotonic acid/vinylneodecanoate copolymer (e.g. RESYN 28 2930), methylvinylether/maleic acid monoalkyl ester copolymers (e.g. GANTREZ® ES 425) and vinylpyrrolidone/vinylcaprolactam/dimethlaminopropylmethacrylic amide copolymer (e.g. AQUAFLEX® SF40).

If the hair fixing polymers contain acid groups, the acid groups are preferably at least partially neutralized with a cosmetically acceptable base. The neutralization degree is preferably from 50 to 100%, especially preferably from 80 to 100%. The neutralization agent can be any organic or inorganic base suitable for cosmetic purposes. For example, aminoalcohols, such as aminomethylpropanol (AMP), triethanolamine or monoethanolamine, and ammonia, NaOH and others are suitable as the bases.

Suitable gel formers (B) include those gel formers which are soluble or dispersible in a C₁- to C₄-alcohol, especially in ethanol or isopropanol and which increase the viscosity of these alcohols when added to them. Salts of fatty acids, diatomaceous earth, polysaccharide ethers, esters of polysaccharides or organo-soluble, especially ethanol-soluble, chitin or chitosan derivatives are suitable. Suitable fatty acid salts include especially salts of stearic acid, such as sodium, potassium or magnesium stearate. Suitable polysaccharide ethers include especially hydroxyalkyl polysaccharides, such as hydroxyalkyl cellulose, hydroxyalkylmethyl cellulose or hydroxyalkyl guar, in which the alkyl groups have from one to four carbon atoms. Hydroxypropyl cellulose is especially preferred as gel former.

Suitable liquid alcohols (C) are monohydric or polyhydric (monovalent or polyvalent) alcohols, which are liquid at room temperature (20° C.) and have one to four carbon atoms. The lower alcohols used for cosmetic purposes, such as ethanol, isopropanol, glycerol, ethylene glycol or propylene glycol, are especially suitable as the alcohol ingredient. Monohydric alcohols with from 2 to 4 carbon atoms, especially ethanol and isopropanol, are particularly preferred. The hair gel is preferably substantially water-free. However it can contain small amounts of water to improve the solubility of other ingredients. Nevertheless the water content must clearly outweigh the alcohol content. The term “substantially water-free” means that the water content is not greater than 10 percent by weight, preferably not greater than 5 percent by weight.

The viscosity of the hair gels according to the invention are preferably from 500 to 50,000 mPa.s, especially preferably from 1,000 to 15,000 mPa.s, according to a dynamic viscosity measurement with a HAAKE Rotation Viscometer VT501 at 25° C. with a test sample according to DIN 53019 (SV-DIN) and a shear rate of 12.9 s⁻¹ (rotation speed stage 5, series B).

The composition according to the invention can also contain additive ingredients suitable and usual for hair treatment compositions, preferably alcohol-soluble additive ingredients, for example wetting agents or emulsifiers from the classes of nonionic, anionic, cationic or amphoteric surface-active agents in an amount of from 0.1 to 15 percent by weight; moisturizing agents; perfume oils, in an amount of from 0.01 to 1 percent by weight; turbidity-inducing agents, such as ethylene glycol distearate, in an amount of about 0.2 to 5.0 percent by weight; pearlescence agents, such as a mixture of fatty acid monoalkylolamide and ethylene glycol distearate, in an amount of about 1.0 to 10 percent by weight; bactericidal and fungicidal agents, such as 2,4,4-trichloro-2-hydroxydiphenyl ether or methyl chloroisothiazoline, in an amount of from 0.01 to 1.0 percent by weight, buffer substances, e.g. sodium citrate or sodium phosphate, in an amount of 0.1 to 1.0 percent by weight; color-imparting substances, e.g. fluorescein sodium salt, in an amount of about 0.1 to 1.0 percent by weight; care materials, such a plant and vegetable extracts, proteins and silk hydrolyzates, lanolin derivatives in an amount of from 0.1 to 5 percent by weight; physiologically compatible silicone derivatives, such as volatile or non-volatile silicone oils or high molecular weight siloxane polymers, in an amount of from 0.05 to 20 percent by weight; light-protective agents, antioxidants, radical trapping agents, luster-imparting agents, vitamins, softeners, combability-improving agents, defatting agents, in an amount of from 0.01 to 4 percent by weight.

The alcoholic gels according to the invention are characterized by a high fixing degree, a rapid drying and a pleasant cooling effect. They can be in a clear form, e.g. using hydroxyalkyl saccharides as thickeners, or in an opaque white form, e.g. using fatty acid salts as thickeners.

The following examples illustrate the above-described invention in more detail, but the details in these examples should not be considered as limiting the claims appended hereinbelow.

### EXAMPLES

### Example 1

Clear Hair Gel

| | | |
|---|---|---|
| 3 | g | Acrylic acid/ethylacrylate/N-tert.-butylacrylate copolymer |
| | | (ULTRAHOLD ® 8) |
| 2 | g | Hydroxypropyl cellulose (KLUCEL ® HF) |
| 0.22 | g | Aminomethylpropanol |
| 0.2 | g | Perfume |
| to 100 | g | Ethanol |

Viscosity: 4600 mPa.s, according to a dynamic viscosity measurement with a HAAKE Rotation viscometer VT501 at 25° C. with a test sample according to DIN 53019 (SV-DIN) and a shear rate of 12.9 s⁻¹ (rotation speed stage 5, series B).

### Example 2

Hair Gel

| | | |
|---|---|---|
| 3 | g | Acrylic acid/ethylacrylate/N-tert.-butylacrylate copolymer |
| | | (ULTRAHOLD ® 8) |
| 7 | g | Hydroxypropyl cellulose (KLUCEL ® HF) |
| 0.22 | g | Aminomethytpropanol |
| 0.2 | g | Perfume |
| to 100 | g | Ethanol |

### Example 3

Hair Gel

| | | |
|---|---|---|
| 5 | g | octylacrylamide/acrylic acid/t-butylaminoethylmeth- |
| | | acrytate/hydroxypropylrflethacrylate copolymer |
| | | (AMPHOMER ®) |
| 2 | g | Hydroxypropyl cellulose (KLUCEL ® HF) |
| 0.52 | g | Aminomethylpropanol |
| 0.2 | g | Perfume |
| to 100 | g | Ethanol |

### Example 4

Hair Gel

| | | |
|---|---|---|
| 10 | g | octylacrylamide/acrylic acid/t-butylaminoethyl meth- |
| | | acrylate/hydroxypropylmethacrylate copolymer |
| | | (AMPHOMER ®) |
| 3 | g | Hydroxypropyl cellulose (KLUCEL ® HF) |
| 0.92 | g | Aminomethyipropanol |
| 0.2 | g | Perfume |
| to 100 | g | Ethanol |

### Example 5

Hair Gel

| | | |
|---|---|---|
| 8 | g | Vinyl acetate/orotonic acid copolymer (LUVISET ® |
| | | CA 66) |
| 5 | g | Hydroxypropyl cellulose (KLUCEL ® HF) |
| 0.66 | g | Aminomethyipropanol |
| 0.2 | g | Perfume |
| to 100 | g | Ethanol |

### Example 6

Hair Gel

| | | |
|---|---|---|
| 5 | g | Vinyl acetate/crotonic acid copolymer (LUVISET ® |
| | | CA 66) |
| 5 | g | t-butylacrylate/ethylacrylate/methacrylic acid |
| | | copolymer (e.g. LUVIMER ® 100 P) |
| 3 | g | Hydroxypropyl cellulose (KLUCEL ® HF) |
| 0.41 | g | Aminomethylpropanol |
| 0.2 | g | Perfume |
| to 100 | g | Ethanol |

### Example 7

Hair Gel

| | | |
|---|---|---|
| 3 | g | Vinyl acetate/crotonic acid copolymer (LUVISET ® |
| | | CA 66) |
| 7 | g | Vinyl acetate/crotonic acid/vinylneodecanoate |
| | | copolymer (RESYN 28 2930) |
| 2 | g | Hydroxypropyl cellulose (KLUCEL ® HF) |
| 0.41 | g | Aminomethylpropanol |
| 0.2 | g | Perfume |
| to 100 | g | Ethanol |

### Example 8

Hair Gel

| | | |
|---|---|---|
| 3 | g | Acrylic acid/ethylacrylate/N-tert.-butylacrylate |
| | | copolymer (ULTRAHOLD ® 8) |
| 2 | g | Hydroxypropylmethyl cellulose (METHOCEL ® 311) |
| 0.22 | g | Aminomethylpropanol |
| 0.2 | g | Perfume |
| 5 | g | Water |
| to 100 | g | Ethanol |

The disclosure in German Patent Application 102 19 597.8 of May 2, 2002 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in water-free hairstyling gels, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A hairstyling gel containing a combination of at least one alcohol-soluble, film-forming and hair-fixing polymer, at least one gel former capable of thickening a liquid alcohol and more than 30 percent by weight of an alcohol with 1 to 4 carbon atoms.

2. The hairstyling gel as defined in claim 1, which is substantially water-free and contains from 0.1 to 20 percent by weight of said at least one alcohol-soluble, film-forming and hair-fixing polymer and from 0.01 to 20 percent by weight of said at least one gel former.

3. The hairstyling gel as defined in claim 1, wherein said at least one alcohol-soluble, film-forming and hair-fixing polymer is selected from the group consisting of polyvinylcaprolactam, vinylpyrrolidone/vinyl acetate copolymers, copolymers of at least one maleic acid monoester and at least one nonionic comonomer, copolymers of at least one acrylic acid ester and at least one nonionic comonomer different from said at least one acrylic acid ester, copolymers of at least one methacrylic acid ester and at least one nonionic comonomer different from said at least one methacrylic acid ester, copolymers of crotonic acid and at least one nonionic comonomer, copolymers of acrylic acid and at least one monomer and copolymers of methacrylic acid and at least one monomer; and wherein said at least one monomer is selected from the group consisting of acrylic acid esters, methacrylic acid esters, acrylamides and methacrylamides.

4. The hairstyling gel as defined in claim 1, wherein said at least one gel former is selected from the group consisting of stearic acid salts, polysaccharide ethers and polysaccharide esters.

5. A gel containing from 1 to 15 percent by weight of at least one alcohol-soluble, film-forming and hair-fixing polymer, from 0.1 to 7 percent by weight of at least one gel former capable of thickening ethanol, from 50 to 98 percent by weight ethanol and from 0 to 10 percent by weight water;
wherein said at least one alcohol-soluble, film-forming and hair-fixing polymer is selected from the group consisting of polyvinylcaprolactam, vinylpyrrolidone/vinyl acetate copolymers, copolymers of at least one maleic acid monoester and at least one nonionic comonomer, copolymers of at least one acrylic acid ester and at least one nonionic comonomer different from said at least one acrylic acid ester, copolymers of at least one methacrylic acid ester and at least one nonionic comonomer different from said at least one methacrylic acid ester, copolymers of crotonic acid and at least one nonionic comonomer, copolymers of acrylic acid and at least one monomer and copolymers of methacrylic acid and at least one monomer; and wherein said at least one monomer is selected from the group consisting of acrylic acid esters, methacrylic acid esters, acrylamides and methacrylamides; and
wherein said at least one gel former is selected from the group consisting of stearic acid salts, hydroxyalkyl celluloses and cellulose esters.
